(19) United States
(12) Patent Application Publication
(10) Pub. No.: US 2019/0091295 A1
(43) Pub. Date: Mar. 28, 2019

(54) Liraglutide in Renal Conditions

(71) Applicant: Novo Nordisk A/S, Bagsvaerd (DK)

(72) Inventor: Soeren Rasmussen, Copenhagen (DK)

(73) Assignee: Novo Nordisk A/S, Bagsvaerd (DK)

(21) Appl. No.: 16/081,461

(22) PCT Filed: Mar. 3, 2017
(86) PCT No.: PCT/EP2017/054977

§ 371 Date: Aug. 31, 2018

(30) Foreign Application Priority Data
Mar. 4, 2016 (EP) 16158738.1

(51) Int. Cl. A61K 38/26; A61K 9/08; A61P 13/12

(52) U.S. Cl. CPC A61K 38/26; A61P 13/12; A61K 9/08

(57) ABSTRACT

The present invention relates to the GLP-1 receptor agonist liraglutide for use in medicine.

The present invention relates to the GLP-1 receptor agonist liraglutide for use in renal conditions in a subject having at least diabetes.

### BACKGROUND

Diabetes is a metabolic disorder characterized by hyperglycaemia that is associated with a high risk of cardiovascular and other serious health-related consequences. A person with diabetes is two to three times more likely to die from cardiovascular causes than people with no history of diabetes, even after controlling for other cardiovascular risk factors. They are also at very high risk of developing serious microvascular complications ultimately leading to premature death: nephropathy and renal failure, retinal disease and blindness, autonomic and peripheral neuropathy, as well as other conditions related to the vascular system: hypertension, lower limb amputation, cognitive decline, and erectile dysfunction.

The majority of people with diabetes have type 2 diabetes, which is characterised by insulin resistance and eventually impaired insulin secretion. Optimal glycaemic control is the treatment goal in subjects with type 2 diabetes, since the risk of long-term complications is increased with poor glycaemic control. Despite the availability of several oral anti-diabetic drugs and insulin, a significant proportion of subjects with type 2 diabetes do not achieve the recommended target levels. With the increasing incidence and prevalence of type 2 diabetes, there is an unmet medical need for treatment alternatives with improved efficacy, safety and convenience.

### SUMMARY

In some embodiments the invention relates to a method of treating type 2 diabetes, comprising administering liraglutide in a therapeutically effective amount to a subject in need thereof, wherein said subject has

(i) one or more vascular diseases selected from the group consisting of cardiovascular disease, cerebrovascular disease, peripheral vascular disease, chronic renal failure, and chronic heart failure, and/or
(ii) one or more risk factors of one or more vascular diseases selected from the group consisting of microalbuminuria, proteinuria, hypertension, left ventricular hypertrophy, left ventricular systolic dysfunction, left ventricular diastolic dysfunction, and ankle/brachial index of <0.9; and
wherein said method reduces or delays nephropathy, macroalbuminuria, increase of serum creatinine, the need for continuous renal-replacement therapy, and/or the progression of moderate renal impairment into end stage renal disease (ESRD).

### BRIEF DESCRIPTION OF DRAWINGS

FIG. 1 shows time to first nephropathy event following administration of liraglutide or placebo.

FIG. 2 shows time to first nephropathy event in subjects with moderate renal impairment following administration of liraglutide or placebo.

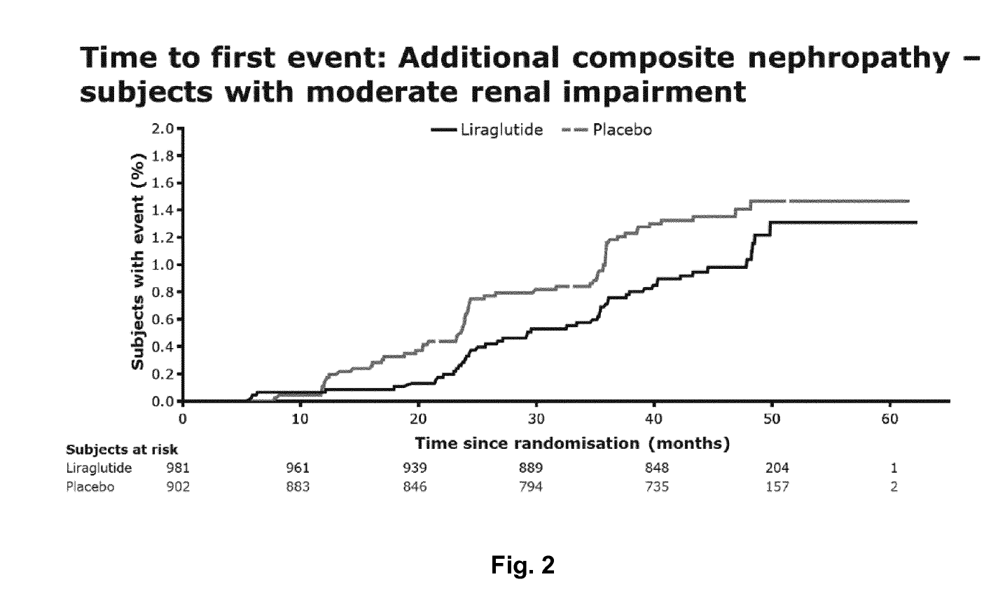

### DESCRIPTION

In some embodiments the invention relates to a method of treating type 2 diabetes, comprising administering liraglutide in a therapeutically effective amount to a subject in need thereof, wherein said subject has

(i) one or more vascular diseases selected from the group consisting of cardiovascular disease, cerebrovascular disease, peripheral vascular disease, chronic renal failure, and chronic heart failure, and/or
(ii) one or more risk factors of one or more vascular diseases selected from the group consisting of microalbuminuria, proteinuria, hypertension, left ventricular hypertrophy, left ventricular systolic dysfunction, left ventricular diastolic dysfunction, and ankle/brachial index of <0.9; and
wherein said method reduces or delays nephropathy, macroalbuminuria, increase of serum creatinine, the need for continuous renal-replacement therapy, and/or the progression of moderate renal impairment into end stage renal disease (ESRD).

In some embodiments the invention relates to a method of reducing of delaying the development of
a. nephropathy,
b. macroalbuminuria,
c. increase of serum creatinine,
d. the need for continuous renal-replacement therapy, and/or
e. the progression of moderate renal impairment into end stage renal disease (ESRD),

said method comprising administering liraglutide in a therapeutically effective amount to a subject in need thereof, wherein the subject has type 2 diabetes and one or more risk factors of vascular disease.

In some embodiments the method reduces or delays nephropathy. In some embodiments the method reduces or delays macroalbuminuria. In some embodiments the method reduces or delays increase of serum creatinine. In some embodiments the method reduces or delays increase of serum creatinine in subjects with eGFR mL/min/1.73 m² per MDRD. In some embodiments the method reduces or delays the need for continuous renal-replacement therapy. In some embodiments the method reduces or delays the progression of moderate renal impairment into end stage renal disease (ESRD).

In some embodiments need for continuous renal-replacement therapy means that the renal impairment of a subject has progressed to ESRD.

In some embodiments the subject has renal impairment. In some embodiments the subject has moderate renal impairment. Renal impairment is usually a chronic disease which progresses from mild renal impairment to end stage renal failure (ESRD). Renal impairment is defined by an eGFR of less than 60 mL/min/1.73 m², e.g. determined by MDRD. Moderate renal impairment is defined by an eGFR in the range of 30-59 mL/min/1.73 m² per MDRD. Severe renal impairment is defined by an eGFR of <30 mL/min/1.73 m² per MDRD. In some embodiments the subject has renal impairment, wherein the estimated glomerular filtration rate (eGFR) is <60, for example <60 mL/min/1.73 m² per Modification of Diet in Renal Disease (MDRD). In some embodiments the subject has eGFR of <60 mL/min/1.73 m² per MDRD. In some embodiments the subject has eGFR of <50 mL/min/1.73 m² per MDRD. In some embodiments the subject has eGFR of <40 mL/min/1.73 m² per MDRD. In some embodiments the subject has eGFR of <30 mL/min/1.73 m² per MDRD. In some embodiments the subject has eGFR of ≥10 mL/min/1.73 m² per MDRD. In some embodiments the estimated glomerular filtration rate (eGFR) is calculated based on serum creatinine concentration followed by either the equation Modification of Diet in Renal Disease (MDRD) or the Chronic Kidney Disease Epidemiology Collaboration (CKD-EPI), both involving variables for age, gender, and race of the subject. eGFR determined by MDRD may be referred to as eGFR-MDRD. eGFR determined by CKD-EPI may be referred to as eGFR-CKD-EPI. The eGFR-MDRD equation may be as defined in formula V: eGFR (mL/min/1.73 m²)=175×(S_{cr})^{-1.154}×(Age)^{-0.203}×(0.742 if female)×(1.212 if African American) [V]. The CKD-EPI equation may be as defined in formula VI: eGFR=141×min^{a}×max^{-1.209}×0.993^{Age}×(1.018 if female)×(1.159 if black) [VI], wherein “min” indicates the minimum of S_{cr}/κ or 1, “max” indicates the maximum of S_{cr}/κ or 1, S_{cr} is serum creatinine in mg/dL, κ is 0.7 for females and 0.9 for males, and α is −0.329 for females or −0.411 for males.

In some embodiments the subject has nephropathy. In some embodiments nephropathy was registered in a subject with one or more symptoms selected from the group consisting of 1) new onset of persistent macroalbuminuria (wherein macroalbuminuria is defined as either: a) 24 hour urine collection above 300 mg albumin, or b) an elevated ratio in a spot sample above 300 mg albumin/g creatinine (to confirm persistent macroalbuminuria, a confirmatory measurement should be performed)); 2) persistent doubling of serum creatinine and creatinine clearance per MDRD≤45 mL/min/1.73 m² (to confirm persistent doubling of serum creatinine, a confirmatory measurement should be performed); 3) need for continuous renal replacement therapy (in the absence of an acute reversible cause); and 4) death due to renal disease.

In some embodiments macroalbuminuria is a urine albumin of ≥300 mg/g creatinine or alternatively as a 24 hour urine collection above 300 mg albumin.

In some embodiments the method reduces or delays nephropathy by 15-40%, such as by 15-30%. In some embodiments the method reduces or delays nephropathy by about 22%. In some embodiments the method reduces or delays nephropathy by 22%. In some embodiments the subject developing nephropathy has a hazard ratio of about 0.78 compared to placebo. In some embodiments the subject developing nephropathy has a hazard ratio of 0.78 with a 95% CI of (0.67; 0.92) compared to placebo.

In some embodiments the method reduces or delays macroalbuminuria by 15-40%, such as by 20-30%. In some embodiments the method reduces or delays macroalbuminuria by about 26%. In some embodiments the method reduces or delays macroalbuminuria by 26%. In some embodiments the subject developing macroalbuminuria has a hazard ratio of about 0.74 compared to placebo. In some embodiments the subject developing macroalbuminuria has a hazard ratio of 0.74 with a 95% CI of (0.60; 0.91) compared to placebo.

In some embodiments the method reduces or delays macroalbuminuria by 15-40%, such as by 20-30%. In some embodiments the method reduces or delays macroalbuminuria by about 26%. In some embodiments the method reduces or delays macroalbuminuria by 26%. In some embodiments the subject developing macroalbuminuria has a hazard ratio of about 0.74 compared to placebo. In some embodiments the subject developing macroalbuminuria has a hazard ratio of 0.74 with a 95% CI of (0.60; 0.91) compared to placebo.

In some embodiments the method reduces or delays increase in serum creatinine by 5-20%, such as by 8-15%. In some embodiments the method reduces or delays increase in serum creatinine by about 12%. In some embodiments the method reduces or delays increase in serum creatinine by 12%. In some embodiments the subject developing increase in serum creatinine has a hazard ratio of about 0.88 compared to placebo.

In some embodiments the method reduces or delays the need for continuous renal-replacement therapy and/or the progression of moderate renal impairment into end stage renal disease (ESRD) by 5-20%, such as by 10-16%. In some embodiments the method reduces or delays the need for continuous renal-replacement therapy and/or the progression of moderate renal impairment into end stage renal disease (ESRD) by about 13%. In some embodiments the method reduces or delays the need for continuous renal-replacement therapy and/or the progression of moderate renal impairment into end stage renal disease (ESRD) by 13%. In some embodiments the subject developing the need for continuous renal-replacement therapy and/or the progression of moderate renal impairment into end stage renal disease (ESRD) has a hazard ratio of about 0.87 compared to placebo.

In some embodiments the invention relates to a method of administering liraglutide in a therapeutically effective amount to a subject, wherein said method reduces or delays nephropathy, macroalbuminuria, increase of serum creatinine, the need for continuous renal-replacement therapy, and/or the progression of moderate renal impairment into ESRD (end stage renal disease). In some embodiments the subject has type 2 diabetes. In some embodiments the subject has type 2 diabetes and said subject has (i) one or more vascular diseases selected from the group consisting of cardiovascular disease, cerebrovascular disease, peripheral vascular disease, chronic renal failure, and chronic heart failure, and/or (ii) at least one risk factors of vascular disease. In some embodiments the method comprises treatment of said type 2 diabetes. In some embodiments the method reduces the risk of developing nephropathy, reduces the risk of developing macroalbuminuria, reduces increase of serum creatinine, reduces the need for continuous renal-replacement therapy, and/or reduces the progression of moderate renal impairment into ESRD (end stage renal disease).

In some embodiments “reduces or delays” when used herein with reference to the method of the invention is “reduces the risk of”. In some embodiments “reduces or delays” when used herein with reference to the method of the invention is “reduces the occurence of”.

### Subject and Subpopulations

The subject to be administered liraglutide according to the present invention may be human, such as an adult human.

The subject to receive liraglutide administration according to the methods of the present invention has type 2 diabetes and has (i) one or more vascular diseases selected from the group consisting of cardiovascular disease, cerebrovascular disease, peripheral vascular disease, chronic renal failure, chronic heart failure, and/or (ii) one or more risk factors of vascular disease. In some embodiments the subject has type 2 diabetes and at least one of cardiovascular disease, cerebrovascular disease, peripheral vascular disease, chronic renal failure, and chronic heart failure. The subject may have type 2 diabetes and cardiovascular disease. The subject may have type 2 diabetes and cerebrovascular disease. The subject may have type 2 diabetes and peripheral vascular disease. The subject may have type 2 diabetes and chronic renal failure. The subject may have type 2 diabetes and chronic heart failure. In some embodiments the subject has type 2 diabetes and one or more risk factors of vascular disease. These vascular diseases may be referred to as concomitant, i.e. one or more vascular diseases are present in the subject at the same time as type 2 diabetes.

In some embodiments said subject has (i) one or more vascular diseases selected from the group consisting of cardiovascular, cerebrovascular, peripheral vascular disease, chronic renal failure, and chronic heart failure, and/or (ii) one or more risk factors of one or more vascular diseases selected from the group consisting of a) microalbuminuria or proteinuria; b) hypertension and/or left ventricular hypertrophy by ECG or imaging; c) left ventricular systolic or diastolic dysfunction by imaging; and d) ankle/brachial index <0.9. In some embodiments said one or more vascular diseases and/or said one or more risk factors of vascular disease were present before initiation of liraglutide administration.

In some embodiments the subject is at least 50 years of age, such as at least 55 years of age or at least 60 years of age.

In some embodiments the subject has HbA_{1c} of at least 7.0%, e.g. prior to receiving liraglutide administration.

In some embodiments the subject is, except for liraglutide, anti-diabetic drug naive or treated with one or more oral anti-diabetic drugs (OADs) or treated with human NPH insulin or long-acting insulin analogue or premixed insulin, alone or in combination with OAD(s). The subject may be anti-diabetic drug naive. The subject may be treated with one or more oral anti-diabetic drugs (OADs). The subject may be treated with human NPH insulin or long-acting insulin analogue or premixed insulin, alone or in combination with OAD(s). In some embodiments the OAD may be selected from the group consisting of sulfonylureas, insulin secretagogues, thiazolidinediones, alpha-glucosidase inhibitors, dipeptidyl peptidase-4 inhibitors, sodium-glucose co-transporter-2 inhibitors, and combinations thereof. In some embodiments the OAD is sulfonylurea (e.g. glimepiride, glipizide, glyburide). In some embodiments the OAD is insulin secretagogues (e.g. biguanides such as metformin or meglitinides such as nateglinide). In some embodiments the OAD is thiazolidinediones (e.g. pioglitazone, rosiglitazone). In some embodiments the OAD is alpha-glucosidase inhibitors (e.g. acarbose, miglitol, voglibose). In some embodiments the OAD is sodium-glucose co-transporter-2 inhibitors (e.g. dapagliflozin, canagliflozin, empagliflozin). In some embodiments the OAD is dipeptidyl peptidase-4 inhibitors (e.g. sitagliptin). In some embodiments the OAD is not a dipeptidyl peptidase-4 inhibitor.

In some embodiments the subject (i) is at least 50 years of age and has at least one of cardiovascular disease, cerebrovascular disease, peripheral vascular disease, chronic renal failure, and chronic heart failure, or (ii) is at least 60 years of age and has one or more risk factors of vascular disease.

In some embodiments the subject a) (i) is at least 50 years of age and has one or more vascular diseases selected from the group consisting of cardiovascular disease, cerebrovascular, disease, peripheral vascular disease, chronic renal failure, and chronic heart failure, or (ii) is at least 60 years of age and has risk factors of vascular disease; b) has HbA_{1c} of at least 7.0%, e.g. at the time prior to receiving liraglutide administration; and c) is anti-diabetic drug naive or treated with one or more oral anti-diabetic drugs (OADs) or treated with human NPH insulin or long-acting insulin analogue or premixed insulin, alone or in combination with OAD(s).

In some embodiments, the cardiovascular disease, cerebrovascular disease, peripheral vascular disease, chronic renal failure, and/or chronic heart failure are selected from the group consisting of: a) myocardial infarction; b) stroke or transient ischaemic attack (TIA); c) coronary, carotid or peripheral arterial revascularisation; d) >50% stenosis on angiography or other imaging of coronary, carotid or lower extremity arteries; e) history of symptomatic coronary heart disease documented by positive exercise stress test or any cardiac imaging, or unstable angina pectoris with ECG (electrocardiogram) changes; f) asymptomatic cardiac ischemia documented by positive nuclear imaging test or exercise test or dobutamine stress echo; g) chronic heart failure NYHA class and h) chronic renal failure, having clinically reached a stage corresponding to a glomerular filtration rate <60 mL/min/1.73 m² per Modification of Diet in Renal Disease (MDRD) or <60 mL/min per Cockroft-Gault formula.

In some embodiments, the subject experienced the a) myocardial infarction; b) stroke or transient ischaemic attack (TIA); or c) coronary, carotid or peripheral arterial revascularisation as a prior event before initiation of liraglutide administration.

The glomerular filtration rate per MDRD may be as defined in formula Ia: GFR (mL/min/1.73 m²)=175 (S_{cr})^{-1.154}×(Age)^{0.203}×(0.742 if female)×(1.212 if African American) [Ib].

The glomerular filtration rate may alternatively be determined by the “Cockroft-Gault formula” may be as defined by Formula III: CrCl (mL/min)=(N×[140-age (years)]×weight*(kg))/Serum creatinine (μM) [III], wherein CrCl is the Cockcroft and Gault creatinine clearance, wherein N is 1.23 for males and 1.04 for females, and wherein if actual weight is greater than 120% IBW then weight is the ideal body weight (IBW) as defined in Formula IIa: IBW (kg)=(no of inches over 5 ft×2.3)+M [IIIa], wherein M is 50 for males and 45.5 for females.

Heart failure exists in different degrees of severity. The most commonly used classification system of heart failure is the New York Heart Association Functional Classification (also referred to as “NYHA”). NYHA categorises subjects in one of four classes I-IV (Table A), based on their degree of limitation during physical activity, and optionally an additional subgroup A-D based on objective assessments, for further details see The Criteria Committee of the New York Heart Association. Nomenclature and Criteria for Diagnosis of Diseases of the Heart and Great Vessels. 9th ed. Boston, Mass.: Little, Brown & Co; 1994:253-256).

TABLE A
NYHA class I-IV criteria

| NYHA | |
|---|---|
| Class | Functional Capacity of the subject |
| I | Subjects with cardiac disease but without resulting limitation of physical |
| | activity. Ordinary physical activity does not cause undue fatigue, palpitation, |
| | dyspnea, or anginal pain. |
| II | Subjects with cardiac disease resulting in slight limitation of physical activity. |
| | They are comfortable at rest. Ordinary physical activity results in fatigue, |
| | palpitation, dyspnea, or anginal pain. |
| III | Subjects with cardiac disease resulting in marked limitation of physical activity. |
| | They are comfortable at rest. Less than ordinary activity causes fatigue, |
| | palpitation, dyspnea, or anginal pain. |
| IV | Subjects with cardiac disease resulting in inability to carry on any physical |
| | activity without discomfort. Symptoms of heart failure or the anginal syndrome |
| | may be present even at rest. If any physical activity is undertaken, discomfort |
| | is increased. |

The cardiovascular disease, cerebrovascular disease, peripheral vascular disease, chronic renal failure, and/or chronic heart failure may be myocardial infarction. The cardiovascular disease, cerebrovascular disease, peripheral vascular disease, chronic renal failure, and/or chronic heart failure may be stroke or prior transient ischaemic attack (TIA). The cardiovascular disease, cerebrovascular disease, peripheral vascular disease, chronic renal failure, and/or chronic heart failure may be coronary, carotid or peripheral arterial revascularisation. The cardiovascular disease, cerebrovascular disease, peripheral vascular disease, chronic renal failure, and/or chronic heart failure may be >50% stenosis on angiography or other imaging of coronary, carotid or lower extremity arteries. The cardiovascular disease, cerebrovascular disease, peripheral vascular disease, chronic renal failure, and/or chronic heart failure may be history of symptomatic coronary heart disease documented by positive exercise stress test or any cardiac imaging, or unstable angina pectoris with ECG changes. The cardiovascular disease, cerebrovascular disease, peripheral vascular disease, chronic renal failure, and/or chronic heart failure may be asymptomatic cardiac ischemia documented by positive nuclear imaging test or exercise test or dobutamine stress echo. The cardiovascular disease, cerebrovascular disease, peripheral vascular disease, chronic renal failure, and/or chronic heart failure may be chronic heart failure NYHA class II-III. The cardiovascular disease, cerebrovascular disease, peripheral vascular disease, chronic renal failure, and/or chronic heart failure may be chronic renal failure, having clinically reached a stage corresponding to a glomerular filtration rate <60 mL/min/1.73 m² per Modification of Diet in Renal Disease (MDRD) or <60 mL/min per Cockroft-Gault formula.

In some embodiments the risk factors of vascular disease may be selected from the group consisting of: a) microalbuminuria or proteinuria; b) hypertension and/or left ventricular hypertrophy by ECG or imaging; c) left ventricular systolic or diastolic dysfunction by imaging; and d) ankle/brachial index <0.9. The risk factors of vascular disease may be microalbuminuria or proteinuria. The risk factors of vascular disease may be hypertension and/or left ventricular hypertrophy by ECG or imaging. The risk factors of vascular disease may be left ventricular systolic or diastolic dysfunction by imaging. The risk factors of vascular disease may be ankle/brachial index <0.9.

In some embodiments the subject has a BMI of at least 30 kg/m². BMI (body mass index) is a measure of body fat based on height and weight. The formula for calculation is BMI=(weight in kilograms)/(height in meters)². In some embodiments the subject has a BMI in the range of 30-50 kg/m². In some embodiments the subject has a BMI of at least 33 kg/m². In some embodiments the subject has a BMI of at least 35 kg/m². In some embodiments the subject has a BMI of at least 37 kg/m². In some embodiments the subject has a BMI of at least 40 kg/m². In some embodiments the subject has a BMI of up to 45 kg/m². In some embodiments the subject has a BMI of up to 40 kg/m².

In some embodiments the subject does not have type 1 diabetes. In some embodiments the subject does not receive administration of a GLP-1 receptor agonist (exenatide or other) or pramlintide or any dipeptidyl peptidase 4 (DPP-4) inhibitor prior to initiating administration of liraglutide according to the present invention. In some embodiments the subject does not receive administration of insulin other than insulin selected from the group consisting of human neutral protamine hagedorn (NPH) insulin, long-acting insulin analogue or premixed insulin. In some embodiments, and in connection with intercurrent illness, the subject receives short-term administration of insulin other than insulin selected from the group consisting of human NPH insulin, long-acting insulin analogue or premixed insulin. Acute decompensation of glycaemic control requiring immediate intensification of treatment to prevent acute complications of diabetes (e.g., diabetic ketoacidosis) in the previous 3 months. In some embodiments the subject does not have an acute coronary or cerebrovascular event in the previous 14 days. In some embodiments the subject does not receive continuous renal replacement therapy. In some embodiments the subject does not have end-stage liver disease. In some embodiments the subject does not have chronic heart failure NYHA IV. In some embodiments the subject does not have a prior solid organ transplant or awaiting solid organ transplant. In some embodiments the subject does not have family or personal history of multiple endocrine neoplasia type 2 (MEN2) or familial medullary thyroid carcinoma (FMTC). In some embodiments the subject does not have personal history of non-familial medullary thyroid carcinoma. In some embodiments the subject does not have malignant neoplasm requiring chemotherapy, surgery, radiation or palliative therapy in the previous 5 years. In some embodiments the subject has intraepithelial squamous cell carcinoma of the skin (Bowen's disease) treated with topical 5-fluorouracil (5FU) and subjects with basal cell skin cancer.

### Liraglutide

Liraglutide is the GLP-1 receptor agonist Arg34,Lys26-(N-epsilon-(gamma-L-glutamyl(N-alfa-hexadecanoyl)))-GLP-1(7-37). Liraglutide may be prepared as described in Example 37 of WO98/08871.

### Pharmaceutical Composition

Liraglutide may be administered in the form of a pharmaceutical composition. The pharmaceutical composition may comprise liraglutide in a concentration from 0.1 mg/ml to 100 mg/ml. In some embodiments the pharmaceutical composition comprises 0.01-50 mg, or 0.01-20 mg, or 0.01-10 mg/ml liraglutide. In some embodiments the pharmaceutical composition comprises 1-20 mg/ml liraglutide.

The pharmaceutical composition may further comprise one or more pharmaceutically acceptable excipients, for example selected from the group consisting of buffer system, preservative, tonicity agent, chelating agent, stabilizer and surfactant. In some embodiments the pharmaceutical composition comprises one or more pharmaceutically acceptable excipients, such as one or more selected from the group consisting of a buffer, an isotonic agent, and a preservative. The formulation of pharmaceutically active ingredients with various excipients is known in the art, see e.g. Remington: The Science and Practice of Pharmacy (e.g. 19th edition (1995), and any later editions). The term “excipient” broadly refers to any component other than the active therapeutic ingredient(s), e.g. liraglutide. The excipient may be an inert substance, an inactive substance, and/or a not medicinally active substance.

In some embodiments the pharmaceutical composition comprises a phosphate buffer, such as a sodium phosphate buffer, e.g. disodium phosphate. In some embodiments the pharmaceutical composition comprises an isotonic agent, such as propylene glycol. In some embodiments the pharmaceutical composition comprises a preservative, such as phenol.

The pharmaceutical composition may be in the form of a solution or a suspension. In some embodiments the pharmaceutical composition is aqueous composition, such as an aqueous solution or an aqueous suspension. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water. An aqueous composition may comprise at least 50% w/w water, or at least 60%, 70%, 80%, or even at least 90% w/w of water. In some embodiments the pharmaceutical composition has a pH in the range of 7.5-9.0.

In some embodiments liraglutide is administered in the form of a pharmaceutical composition comprising about 1-20 mg/ml liraglutide, about 2-15 mM phosphate buffer, about 2-25 mg/ml propylene glycol, about 1-18 mg/ml phenol, and has a pH in the range of 7.5-9.0. In some embodiments liraglutide is administered in the form of a pharmaceutical composition comprising about 6 mg/ml liraglutide, about 1.42 mg/ml disodium phosphate dihydrate, about 14.0 mg/ml propylene glycol, about 5.5 mg/ml phenol, and has pH of about 8.15. In some embodiments liraglutide is administered in the form of a pharmaceutical composition comprising 6 mg/ml liraglutide, 1.42 mg/ml disodium phosphate dihydrate, 14.0 mg/ml propylene glycol, 5.5 mg/ml phenol, and has pH of 8.15.

### Administration Regimen

In some embodiments liraglutide is administered as a chronic treatment for at least 10 months. In some embodiments liraglutide is administered as a chronic treatment for at least 12 months. In some embodiments liraglutide is administered as a chronic treatment for at least 12 months. In some embodiments liraglutide is administered as a chronic treatment for at least 15 months. In some embodiments liraglutide is administered as a chronic treatment for at least 18 months. In some embodiments liraglutide is administered as a chronic treatment for up to 36 months.

Liraglutide may be administered in a therapeutically effective amount, such as an amount therapeutically effective to treat type 2 diabetes. The therapeutically effective amount of liraglutide can be assessed by a medical doctor. The dosage of liraglutide may be in the range from 0.1 to 10 mg.

Liraglutide may be administered once daily. In some embodiments liraglutide is administered once daily at any time in the day. In some embodiments the daily dosage of liraglutide is in the range from 0.4 to 4.0 mg, such as in the range from 0.4 to 2.0 mg.

In some embodiments the daily dosage of liraglutide is selected from the group consisting of 0.6, 1.2, and 1.8 mg. In some embodiments the daily dosage of liraglutide is 3.0 mg.

In some embodiments the term “chronic treatment” as used herein with reference to liraglutide means administration in an amount and frequency to provide a therapeutic effect. In some embodiments the term “chronic treatment” as used herein with reference to liraglutide means once daily administration 0.4-4.0 mg, such as 0.6, 1.2, or 1.8 mg, liraglutide.

Liraglutide may be administered via parenteral administration, for example subcutaneous injection. Liraglutide may be administered using a pen-injector, such as a 3 ml disposable pen-injector.

Unless otherwise states, ranges herein include their end points. In some embodiments the term “a” means “one or more”. In some embodiments, terms presented in singular form also include the plural situation. Herein the term “about” means±10% of the value referred to, and includes the value.

### Embodiments of the Invention

Non-limiting embodiments of the invention include:
1. A method for reducing of delaying the development of
   f. nephropathy,
   g. macroalbuminuria,
   h. increase of serum creatinine,
   i. the need for continuous renal-replacement therapy, and/or
   j. the progression of moderate renal impairment into end stage renal disease (ESRD),
said method comprising administering liraglutide in a therapeutically effective amount to a subject in need thereof, wherein the subject has type 2 diabetes and one or more risk factors of vascular disease.
2. A method of administering liraglutide in a therapeutically effective amount to a subject, wherein said method reduces or delays
   a. nephropathy,
   b. macroalbuminuria,
   c. increase of serum creatinine,
   d. the need for continuous renal-replacement therapy, and/or
   e. the progression of moderate renal impairment into ESRD (end stage renal disease).
3. A method of administering liraglutide in a therapeutically effective amount to a subject, wherein said method
   a. reduces the risk of developing nephropathy,
   b. reduces the risk of developing macroalbuminuria,
   c. reduces increase of serum creatinine,
   d. reduces the need for continuous renal-replacement therapy, and/or
   e. reduces the progression of moderate renal impairment into ESRD (end stage renal disease).
4. A method of treating type 2 diabetes, comprising administering liraglutide in a therapeutically effective amount to a subject in need thereof, wherein said subject has
   (i) one or more vascular diseases selected from the group consisting of cardiovascular disease, cerebrovascular disease, peripheral vascular disease, chronic renal failure, and chronic heart failure, and/or
   (ii) one or more risk factors of one or more vascular diseases selected from the group consisting of microalbuminuria, proteinuria, hypertension, left ventricular hypertrophy, left ventricular systolic dysfunction, left ventricular diastolic dysfunction, and ankle/brachial index of <0.9; and
wherein said method reduces or delays
   a. nephropathy,
   b. macroalbuminuria,
   c. increase of serum creatinine,
   d. the need for continuous renal-replacement therapy, and/or
   e. the progression of moderate renal impairment into end stage renal disease (ESRD).
5. A method of administering liraglutide in a therapeutically effective amount to a subject, wherein said method reduces the risk of developing nephropathy.
6. A method of administering liraglutide in a therapeutically effective amount to a subject, wherein said method reduces the risk of developing macroalbuminuria.
7. The method according to any one of the preceding embodiments, wherein said method reduces increase of serum creatinine.
8. A method of administering liraglutide in a therapeutically effective amount to a subject, wherein said method reduces the need for continuous renal-replacement therapy.
9. A method of administering liraglutide in a therapeutically effective amount to a subject, wherein said method reduces the progression of moderate renal impairment into ESRD.
10. The method according to any one of the preceding embodiments, wherein said subject has moderate renal impairment.
11. The method according to any one of the preceding embodiments, wherein said method reduces the need for continuous renal-replacement therapy, and wherein said subject has moderate renal impairment.
12. The method according to any one of the preceding embodiments, wherein moderate renal impairment is eGFR-MDRD in the range from more than 30 to less than 60 mL/min/1.73 m².
13. The method according to any one of the preceding embodiments, wherein liraglutide is administered as a chronic treatment for at least 10 months.
14. The method according to any one of the preceding embodiments, wherein liraglutide is administered as a chronic treatment for at least 12 months.
15. The method according to any one of the preceding embodiments, wherein liraglutide is administered as a chronic treatment for at least 12 months.
16. The method according to any one of the preceding embodiments, wherein liraglutide is administered as a chronic treatment for at least 15 months.
17. The method according to any one of the preceding embodiments, wherein liraglutide is administered as a chronic treatment for at least 18 months.
18. The method according to any one of the preceding embodiments, wherein liraglutide is administered as a chronic treatment for up to 36 months.
19. The method according to any one of the preceding embodiments, wherein said subject has type 2 diabetes.
20. The method according to any one of the preceding embodiments, wherein said subject has type 2 diabetes and said subject has (i) one or more vascular diseases selected from the group consisting of cardiovascular disease, cerebrovascular disease, peripheral vascular disease, chronic renal failure, and chronic heart failure, and/or (ii) at least one risk factors of vascular disease.
21. The method according to any one of the preceding embodiments, wherein said method comprises treatment of said type 2 diabetes.
22. The method of according to any one of the preceding embodiments, wherein said subject has (i) one or more vascular diseases selected from the group consisting of cardiovascular, cerebrovascular, peripheral vascular disease, chronic renal failure, and chronic heart failure, and/or (ii) one or more risk factors of one or more vascular diseases selected from the group consisting of a) microalbuminuria or proteinuria; b) hypertension and/or left ventricular hypertrophy by ECG or imaging; c) left ventricular systolic or diastolic dysfunction by imaging; and d) ankle/brachial index <0.9.
23. The method according to any one of the preceding embodiments, wherein said one or more vascular diseases and/or said one or more risk factors of vascular disease were present before initiation of liraglutide administration.
24. The method according to any one of the preceding embodiments, wherein liraglutide is administered once daily.
25. The method according to any one of the preceding embodiments, wherein liraglutide is administered once daily in an amount in the range of 0.4-4.0 mg per day, such as 0.6, 1.2, or 1.8 mg per day.
26. The method according to any one of the preceding embodiments, wherein liraglutide is administered in the form of a pharmaceutical composition comprising about 1-20 mg/ml liraglutide, about 2-15 mM phosphate buffer, about 2-25 mg/ml propylene glycol, about 1-18 mg/ml phenol, and has a pH in the range of 7.5-9.0.
27. The method according to any one of the preceding embodiments, wherein liraglutide is administered in the form of a pharmaceutical composition comprising about 6 mg/ml liraglutide, about 1.42 mg/ml disodium phosphate dihydrate, about 14.0 mg/ml propylene glycol, about 5.5 mg/ml phenol, and has pH of about 8.15.
28. The method according to any one of the preceding embodiments, wherein liraglutide is administered in the form of a pharmaceutical composition comprising 6 mg/ml liraglutide, 1.42 mg/ml disodium phosphate dihydrate, 14.0 mg/ml propylene glycol, 5.5 mg/ml phenol, and has pH of 8.15.

### Examples

### List of Abbreviations

MACE: Major adverse cardiovascular event

HbA_{1c}: Glycosylated haemoglobin

GLP-1: Glucagon-like peptide-1

BMI: Body mass index

CV: cardiovascular

OAD: Oral antidiabetic drug

TIA: transient ischaemic attack

CI: Confidence interval

Clinical trial: Materials and Methods

A long-term, multi-centre, international, randomised double-blind, placebo-controlled trial with 9340 human subjects was carried out with treatment for at least 3.5 years and up to 5 years per subject; and this trial concerned the incidence of cardiovascular events in adult human subjects with type 2 diabetes that were at high risk for cardiovascular events, including such subjects with existing cardiovascular disease. The primary objective of this trial was to determine the long term effect of treatment with liraglutide compared to placebo on cardiovascular events in subjects with type 2 diabetes. The secondary objective was to assess the efficacy and safety with regard to clinically important events or other surrogate parameters of treatment with liraglutide compared to placebo in adults with type 2 diabetes that were at high risk for cardiovascular events. All trial endpoints were collected and assessed throughout the entire duration of the trial. Subject inclusion and exclusion criteria were as described in Table 2. The subject's characteristics, cardiovascular risk profile, renal function, cardiovascular medication, and antidiabetic treatment regimens of the randomised subjects at baseline were as shown in Tables 3a-e. Overall trial duration was planned as 18 months of recruitment period followed by 42 months of treatment from last subject randomised. The trial started with an open-label run-in period of two weeks with placebo following which subjects were randomised in a 1:1 manner to receive liraglutide or placebo as an add-on to their standard of care (SOC) treatment. The subject's SOC treatment was as shown in Table 4. After randomisation, treatment with liraglutide or placebo was double-blind throughout the trial. Subjects were started on 0.6 mg of liraglutide or placebo. The term “placebo” as used herein refers to a formulation identical to the liraglutide formulation except not comprising liraglutide and the placebo was administered in the volume used in the equivalent liraglutide dosage. Dose escalation of liraglutide or placebo proceeded to 1.2 mg after one week followed by dose escalation to 1.8 mg after one week. After the dose escalation, 95% of subjects received 1.8 mg of liraglutide or placebo, 5% of subjects received 1.2 mg of liraglutide or placebo, and 5% of subjects received 0.6 mg of liraglutide or placebo. Dose increase period could be extended if required in view of a subject's tolerance to the trial product (i.e. liraglutide or placebo). The dosage could be reduced at any time in the trial if required by the subject's tolerance to the trial product. Subjects received liraglutide or placebo by subcutaneous administrations once daily in addition to the subject's standard treatment at a maximum dose of 1.8 mg liraglutide or placebo. The subcutaneous injection was made either in the abdomen, thigh or upper arm. The formulations were administered in the form of an aqueous solution comprising liraglutide or placebo, both using a 3 ml disposable pen-injector. This pen-injector was identical for the liraglutide and placebo administrations. This aqueous solution contained 6.0 mg/ml liraglutide, 1.42 mg/ml disodium phosphate dihydrate, 14.0 mg/ml propylene glycol, 5.5 mg/ml phenol, and had pH 8.15. Liraglutide may be prepared as described in WO98/08871.

The term “baseline” herein (e.g. used as part of “baseline characteristics” or “baseline cardiovascular risk profile”) may refer to the level of a certain parameter (e.g. level of HbA1c) by the determination made in connection with the medical visit at the time of randomisation of the subject. In some embodiments the term baseline refers to a parameter before initiating administration of liraglutide, e.g. the history of a certain event in a subject.

The results of this trial may be presented herein as a number or fraction of subjects experiencing an event. Alternatively, the results of this trial may be presented with hazard ratios estimated in a Cox proportional hazard model, which is the standard statistical model used for estimating time to an event. The term “hazard ratio” (also referred to as “HR”) as used herein means the instantaneous risk ratio of experiencing an event when administered liraglutide compared to placebo which are the two treatments in this trial. An upper limit of the 95% confidence interval (CI) for the HR of less than 1.00 means that the estimated treatment ratio between liraglutide and placebo with respect to the event of interests is statistically significant in favour of liraglutide on a 5% significance level. A 5% significance level is the standard level for investigating significance in clinical trials. For example, a HR value of 0.78 for time to first CV death with a 95% CI of (0.66; 0.94) means that liraglutide provides an estimated 22% risk reduction of experiencing CV death at any given point in time compared to placebo and this risk reduction is statistically significant because 0.94 is less than 1.00.

TABLE 2
Subject inclusion and exclusion criteria (all inclusion criteria were fulfilled for
eligible subjects; one or more exclusion criteria were fulfilled for subjects to be excluded;
however, 150 patients violated at least one inclusion or exclusion criteria)

| Inclusion Criteria | Exclusion Criteria |
|---|---|
| Men or women with type 2 diabetes | Type 1 diabetes |
| Age ≥ 50 years at screening and | Use of a GLP-1 receptor agonist |
| concomitant cardiovascular disease, | (exenatide, liraglutide or other) or |
| cerebrovascular disease, peripheral | pramlintide or any dipeptidyl |
| vascular disease, chronic renal failure, | peptidase 4 (DPP-4) inhibitor within |
| and/or chronic heart failure selected from | the 3 months prior to screening (trial |
| the group consisting of: a) prior | start) |
| myocardial infarction; b) prior stroke or | Use of insulin other than human |
| prior transient ischaemic attack (TIA); c) | neutral protamine hagedorn (NPH) |
| prior coronary, carotid or peripheral | insulin or long-acting insulin analogue |
| arterial revascularisation; d) >50% | or premixed insulin within 3 months |
| stenosis on angiography or other imaging | prior to screening. Short-term use of |
| of coronary, carotid or lower extremity | other insulin during this period in |
| arteries; e) history of symptomatic | connection with intercurrent illness |
| coronary heart disease documented by | was allowed, at Investigator's |
| positive exercise stress test or any | discretion |
| cardiac imaging, or unstable angina | Acute decompensation of glycaemic |
| pectoris with ECG (electrocardiogram) | control requiring immediate |
| changes; f) asymptomatic cardiac | intensification of treatment to prevent |
| ischemia documented by positive nuclear | acute complications of diabetes (e.g., |
| imaging test or exercise test or | diabetic ketoacidosis) in the previous |
| dobutamine stress echo; g) chronic heart | 3 months |
| failure NYHA class II-III; and h) chronic | An acute coronary or cerebrovascular |
| renal failure, having clinically reached a | event in the previous 14 days |
| stage corresponding to a glomerular | Current continuous renal replacement |
| filtration rate < 60 mL/min/1.73 m² per | therapy |
| Modification of Diet in Renal Disease | End-stage liver disease |
| (MDRD) or < 60 mL/min per Cockroft- | Chronic heart failure NYHA IV |
| Gault formula; wherein “prior” refers to | A prior solid organ transplant or |
| before initiation of liraglutide | awaiting solid organ transplant |
| administration; OR age ≥ 60 years at | Family or personal history of multiple |
| screening and other specified risk factors | endocrine neoplasia type 2 (MEN2) or |
| of one or more vascular diseases selected | familial medullary thyroid carcinoma |
| from the group consisting of: a) | (FMTC) |
| microalbuminuria or proteinuria; b) | Personal history of non-familial |
| hypertension and/or left ventricular | medullary thyroid carcinoma |
| hypertrophy by ECG or imaging; c) left | Malignant neoplasm requiring |
| ventricular systolic or diastolic | chemotherapy, surgery, radiation or |
| dysfunction by imaging; and d) | palliative therapy in the previous 5 |
| ankle/brachial index < 0.9 | years. Subjects with intraepithelial |
| HbA_{1c} ≥ 7.0% at screening | squamous cell carcinoma of the skin |
| Anti-diabetic drug naive or treated with | (Bowen's disease) treated with topical |
| one or more oral anti-diabetic drugs | 5-fluorouracil (5FU) and subjects with |
| (OADs) or treated with human NPH | basal cell skin cancer are allowed to |
| insulin or long-acting insulin analogue or | enter the trial |
| premixed insulin, alone or in combination | |
| with OAD(s) | |

TABLE 3a
Baseline characteristics

| | Liraglutide | Placebo |
|---|---|---|
| Number of subjects | 4668 | 4672 |
| Male sex, N (%) | 3011 (64.5) | 2992 (64.0) |
| Age, years | 64.2 | 64.4 |
| Diabetes duration, years | 12.8 | 12.9 |
| HbA_{1c}, % | 8.7 | 8.7 |
| BMI, kg/m² | 32.5 | 32.5 |
| Body weight, kg | 91.9 | 91.6 |
| Systolic blood pressure, mmHg | 135.9 | 135.9 |
| Diastolic blood pressure, mmHg | 77.2 | 77.0 |
| Heart failure*, N (%) | 832 (17.8) | 821 (17.6) |

Full analysis set;
data are mean unless stated otherwise.
*Heart failure includes NYHA class I, II and III.
%: proportion of subjects.
BMI: body mass index.
HbA1c: glycosylated haemoglobin.
NYHA: New York Heart Association.

TABLE 3b
Cardiovascular risk profile at baseline

| | Liraglutide | | Placebo | |
|---|---|---|---|---|
| | N | % | N | % |
| Number of subjects | 4668 | | 4672 | |
| Age ≥ 50 years and established CV | 3815 | 81.7 | 3749 | 80.2 |
| disease | | | | |
| Prior myocardial infarction | 1464 | 31.4 | 1400 | 30.0 |
| Prior stroke or prior TIA | 730 | 15.6 | 777 | 16.6 |
| Prior arterial revascularisation | 1766 | 37.8 | 1719 | 36.8 |
| >50% stenosis on angiography | 1188 | 25.4 | 1191 | 25.5 |
| Documented history of symptomatic | 412 | 8.8 | 406 | 8.7 |
| coronary heart disease | | | | |
| Documented asymptomatic cardiac | 1241 | 26.6 | 1231 | 26.3 |
| ischaemia | | | | |
| Chronic heart failure NYHA II or III | 653 | 14.0 | 652 | 14.0 |
| Chronic kidney failure | 1185 | 25.4 | 1122 | 24.0 |
| Age ≥ 60 years and risk factors for CV | 853 | 18.3 | 923 | 19.8 |
| disease | | | | |
| Microalbuminuria or proteinuria | 504 | 10.8 | 560 | 12.0 |
| Hypertension and/or left ventricular | 249 | 5.3 | 253 | 5.4 |
| hypertrophy | | | | |
| Left ventricular systolic or diastolic | 204 | 4.4 | 191 | 4.1 |
| dysfunction | | | | |
| Ankle/brachial index < 0.9 | 126 | 2.7 | 134 | 2.9 |

Full analysis set.
*Chronic kidney failure was defined as having clinically reached a stage corresponding to eGFR < 60 mL/min/1.73 m² per MDRD or < 60 mL/min per Cockroft-Gault formula, reported at the discretion of the investigator.
%: proportion of subjects.
CV: cardiovascular.
eGFR: estimated glomerular filtration rate.
MDRD: modification of diet in renal disease.
N: number of subjects.
NYHA: New York Heart Association.
TIA: transient ischaemic attack.

TABLE 3c
Renal function at baseline

| | Liraglutide | | Placebo | |
|---|---|---|---|---|
| | N | % | N | % |
| Number of subjects | 4668 | 100.0 | 4672 | 100.0 |
| Normal renal function (eGFR ≥ 90) | 1620 | 34.7 | 1655 | 35.4 |
| Mild impairment (eGFR 60-89) | 1932 | 41.4 | 1975 | 42.3 |
| Moderate impairment (eGFR 30-59) | 999 | 21.4 | 935 | 20.0 |
| Severe impairment (eGFR < 30) | 117 | 2.5 | 107 | 2.3 |

Full analysis set.
eGFR (mL/min/1.73 m2) as per MDRD formula.
%: proportion of subjects.
eGFR: estimated glomerular filtration rate;
MDRD: modification of diet in renal disease.
N: number of subjects.

TABLE 3d
Cardiovascular medication at baseline

| | Liraglutide | | Placebo | |
|---|---|---|---|---|
| | N | % | N | % |
| Number of subjects | 4668 | | 4672 | |
| Antihypertensive therapy | 4322 | 92.6 | 4299 | 92.0 |
| Beta blockers | 2649 | 56.7 | 2524 | 54.0 |
| Calcium channel blockers | 1531 | 32.8 | 1477 | 31.6 |
| ACE inhibitors and ARB | 3898 | 83.5 | 3833 | 82.0 |
| Others | 468 | 10.0 | 452 | 9.7 |
| Diuretics | 1950 | 41.8 | 1949 | 41.7 |
| Loop diuretics | 823 | 17.6 | 833 | 17.8 |
| Others | 1405 | 30.1 | 1392 | 29.8 |
| Lipid-lowering drugs | 3554 | 76.1 | 3511 | 75.1 |
| Statins | 3395 | 72.7 | 3334 | 71.4 |
| Others | 655 | 14.0 | 676 | 14.5 |
| Platelet aggregation inhibitors | 3203 | 68.6 | 3119 | 66.8 |
| Acetylsalicylic acid | 2975 | 63.7 | 2899 | 62.1 |
| Others | 718 | 15.4 | 743 | 15.9 |
| Other anti-thrombotic medication | 309 | 6.6 | 314 | 6.7 |

Full analysis set.
83 subjects had missing initiation drug date, these were assumed to be on treatment at baseline.
%: proportion of subjects.
ACE: angiotensin converting enzyme.
ARB: angiotensin receptor blocker.
N: number of subjects.

TABLE 3e
Antidiabetic treatment regimens at baseline

| | Liraglutide | | Placebo | |
|---|---|---|---|---|
| | N | % | N | % |
| Number of subjects | 4668 | | 4672 | |
| Insulin-naïve | 2633 | 56.4 | 2548 | 54.5 |
| Not on treatment* | 196 | 4.2 | 170 | 3.6 |
| OADs only | 2437 | 52.2 | 2378 | 50.9 |
| Insulin treatment | 2035 | 43.6 | 2124 | 45.5 |
| Insulin only | 361 | 7.7 | 376 | 8.0 |
| Insulin + OADs | 1674 | 35.9 | 1748 | 37.4 |

Full analysis set.
*Includes subjects not on insulin/OAD and subjects on no pharmacologic treatment.
%: proportion of subjects.
N: number of subjects.
OAD: oral antidiabetic drug.

TABLE 4
Standard of care guidelines for subjects in this trial

| Parameter | Standard of care guideline |
|---|---|
| Blood glucose | HbA1c ≤ 7.0% (individualized dependin0 g on patient). If > 7.0%, |
| | additional HbA1c measurement after 3 m. If HbA1c still > 7.0%, |
| | treatment was intensified to achieve target if appropriate. |
| Therapy | Lifestyle modifications and metformin are considered foundational |
| | therapy in most countries of this trial |
| Intensification | Add-on therapy: thiazolidinediones, sulfonylureas, α-glucosidase |
| | inhibitors, according to local labels (dipeptidyl peptidase-4 inhibitors |
| | and other incretin based therapies were not allowed). Insulin therapy |
| | should be based on local practice, including basal, basal/bolus, |
| | premix, and mealtime bolus (SIT). |
| Blood pressure | Target: 130/80 mmHg. |
| Antihypertensive | First line: ACE inhibitors or ARBs. Based on individual patient needs: |
| therapy | Ca²⁺-blockers, diuretics, others. |
| Lipid targets and | LDL < 100 mg/dL (<70 mg/dL in patients with previous |
| therapy | cardiovascular events). Statins recommended for all patients. |
| | Second line therapy at investigator discretion. |
| Antiplatelet | Aspirin or clopidogrel (if aspirin intolerant) for patients with prior |
| therapy | cardiovascular events (MI, cerebrovascular accident, or |
| | revascularization). |

### Results: Renal Effects of Liraglutide

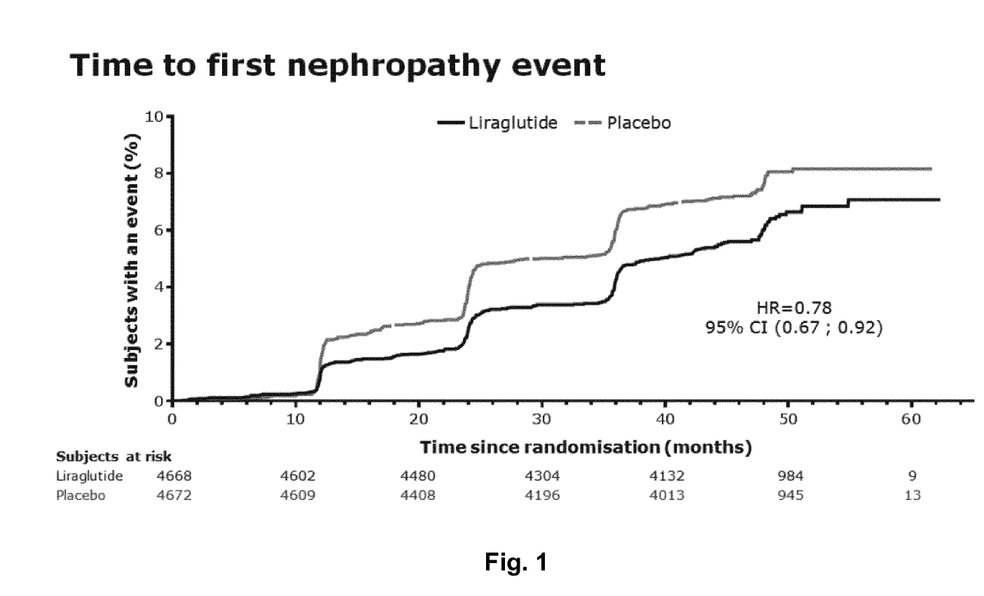

The results of the trial with respect to renal effects are shown in Tables 5-6 and FIG. 1.

TABLE 5
Subjects with moderate renal impairment

| | Liraglutide | | Placebo | |
|---|---|---|---|---|
| | N | % | N | % |
| Number of subjects with moderate renal | 981 | | 902 | |
| impairment | | | | |
| Nephropathy in subjects with moderate renal | 48 | 4.9 | 62 | 6.9 |
| impairment | | | | |
| Doubling of serum creatinine | 26 | | 32 | |
| Need for continuous renal-replacement therapy | 20 | | 29 | |
| Death due to renal disease | 2 | | 1 | |

TABLE 6
Subjects with renal impairment at any stage

| | Hazard ratio | Liraglutide | | Placebo | |
|---|---|---|---|---|---|
| | (95% CI) | N | % | N | % |
| Number of subjects (FAS) | | 4668 | | 4672 | |
| Nephropathy at any stage, | 0.78 (0.67; 0.92) | 268 | 5.7 | 337 | 7.2 |
| EAC-confirmed | | | | | |
| New onset of persistent | 0.74 (0.60; 0.91) | 161 | 3.4 | 215 | 4.6 |
| macroalbuminuria* | | | | | |
| Persistent doubling of | 0.88 (0.66; 1.18) | 87 | 1.9 | 97 | 2.1 |
| serum creatinine clearance | | | | | |
| and eGFR ≤ 45 mL/min/ | | | | | |
| 1.73 m² per MDRD | | | | | |
| Need for continuous renal- | 0.87 (0.61; 1.24) | 56 | 1.2 | 64 | 1.4 |
| replacement therapy | | | | | |
| Death due to renal disease | 1.59 (0.52; 4.87) | 8 | 0.2 | 5 | 0.1 |

These results show that liraglutide reduces the risk of developing renal impairment, such as nephropathy, compared to placebo. This effect is particularly pronounced in subjects with moderate renal impairment. The need for continuous renal-replacement therapy shows that the subject has end stage renal disease (ESRD).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-18. (canceled)

19. A method for reducing the progression of moderate renal impairment into end stage renal disease (ESRD),
comprising administering liraglutide in a therapeutically effective amount to a subject in need thereof,
wherein the subject has type 2 diabetes and moderate renal impairment,
wherein the moderate renal impairment is an Estimated Glomerular Filtration Rate determined by Modification of Diet in Renal Disease (eGFR-MDRD) in the range of 30 to less than 60 mL/min/1.73 m².

20. The method according to claim 19, wherein liraglutide is administered as a chronic treatment for at least 12 months.

21. The method according to claim 19, wherein liraglutide is administered once daily.

22. The method according to claim 19, wherein liraglutide is administered once daily in an amount in the range of 0.4-4.0 mg per day.

23. The method according to claim 22, wherein liraglutide is administered in the form of a pharmaceutical composition comprising about 1-20 mg/ml liraglutide, about 2-15 mM phosphate buffer, about 2-25 mg/ml propylene glycol, about 1-18 mg/ml phenol, and has a pH in the range of 7.5-9.0.

24. The method according to claim 23, wherein liraglutide is administered in the form of a pharmaceutical composition comprising about 6 mg/ml liraglutide, about 1.42 mg/ml disodium phosphate dihydrate, about 14.0 mg/ml propylene glycol, about 5.5 mg/ml phenol, and has a pH of about 8.15.

25. The method according to claim 24, wherein liraglutide is administered in the form of a pharmaceutical composition comprising 6 mg/ml liraglutide, 1.42 mg/ml disodium phosphate dihydrate, 14.0 mg/ml propylene glycol, 5.5 mg/ml phenol, and has a pH of 8.15.

26. The method according to claim 19, wherein liraglutide is administered once daily at 0.6 mg per day.

27. The method according to claim 19, wherein liraglutide is administered once daily at 1.2 mg per day.

28. The method according to claim 19, wherein liraglutide is administered once daily at 1.8 mg per day.
